(19) United States
(12) Patent Application Publication
(10) Pub. No.: US 2015/0065426 A1
(43) Pub. Date: Mar. 5, 2015

(54) Testosterone Booster Transdermal Compositions

(71) Applicants: Tsu-I Catherine Wang, Sugar Land, TX (US); Bruce Vincent Biundo, Houston, TX (US)

(72) Inventors: Tsu-I Catherine Wang, Sugar Land, TX (US); Bruce Vincent Biundo, Houston, TX (US)

(73) Assignee: Professional Compounding Centers Of America, Houston, TX (US)

(21) Appl. No.: 14/011,541

(22) Filed: Aug. 27, 2013

(51) Int. Cl. A61K 47/44; A61K 31/4196; A61K 47/14; A61K 47/24; A61K 47/12; A61K 31/568; A61K 38/24

(52) U.S. Cl. CPC A61K 47/44; A61K 31/568; A61K 31/4196; A61K 38/24; A61K 47/24; A61K 47/12; A61K 47/14
USPC 514/10.2

(57) ABSTRACT

Formulations and methods for transdermal drug delivery compositions that include synergistic combination of three pharmaceutical active ingredients (APIs), such as testosterone, anastrozole, and HCG are disclosed. TBTC of the present disclosure may be indicated for reducing symptoms of testosterone deficiency. Disclosed TBTC may include permeation enhancers that may improve penetration of testosterone, anastrozole, and HCG in human skin. Permeation enhancer compositions within TBTC may include oils from amazon rainforest such as Pracaxi oil, Plukenetia volubilis seed oil, Inaja oil, and Patauá oil, which includes behenic and oleic fatty acids that may provide penetration power. TBTC may include organic solvents as transdermal penetration enhancers. Additionally, TBTC may include physiological lipids, phospholipids, and one or more butters rich in linoleic acid and linolenic acid that may also provide penetration power with restorative benefits to the skin.

### CROSS-REFERENCE TO RELATED APPLICATIONS

The disclosure here described is a sister application of the disclosure referenced as PCC-PHTH-088, titled “Transdermal Delivery of Anastrozole for Systemic Effect”.

### BACKGROUND

1. Field of the Disclosure

The present disclosure relates in general to transdermal delivery of testosterone boosters; and more particularly, to compositions and methods for transdermal testosterone booster compositions.

2. Background Information

Testosterone is the primary male androgen, playing a vital role in overall male health. Testosterone is essential to the development and maintenance of specific reproductive tissues (testes, prostate, epididymis, seminal vesicle, and penis) and male secondary sex characteristics. Testosterone plays a key role in libido and erectile function and is necessary for the initiation and maintenance of spermatogenesis. Testosterone also has important functions not related to reproductive tissues. For example, testosterone positively affects body composition by increasing nitrogen retention, which supports lean body mass, muscle size and strength. Testosterone may also act on bone to stimulate bone formation.

Several forms of testosterone therapy exist in the United States today. Recently, transdermal preparations have gained favor in the market. However, a scrotal testosterone patch results in supraphysiologic levels of 5α-dihydrotestosterone (DHT) due to the high concentration of 5α-reductase in scrotal skin. It is not known whether these elevated DHT levels have any long-term health consequences. Nonscrotal systems are considered more convenient and most patients achieve average serum concentrations within the normal range and have normal levels of DHT. Oral testosterone therapy is not recommended because doses required for replacement therapy are associated with significant risk of hepatotoxicity.

Furthermore Human Chorionic Gonadotropin (HCG) in males, like luteinizing hormone (LH) already produced in the body, stimulates Leydig cells of the testes to produce the male hormone testosterone. HCG may be used to avoid testicular shrinkage, may not suppress FSH production, and therefore can preserve male fertility. HCG is generally given as an injection under the skin or intramuscularly. HCG may increase testosterone production in men as well as sperm count.

Testosterone replacement therapy may trigger the hypothalamus to shut down its production of GnRH (gonadotropin releasing hormone). Without GnRH, the pituitary gland stops releasing LH. Without LH the testes (testicles or gonads) shut down their production of testosterone. For males HCG closely resembles LH. If the testicles have shrunken after long-term testosterone use, testicles may begin to enlarge and start their testosterone production shortly after HCG therapy is instituted. HCG allows testes to produce testosterone and to increase their size.

Anastrozole is an aromatase inhibitors (AIs) that works by binding to the aromatase enzyme that converts testosterone into estrogen. Thus anastrozole effectively inhibits or blocks conversion of testosterone into estrogen, which leads to the increase of testosterone levels. Doctors are now prescribing anastrozole in conjunction with HCG.

Transdermal drug delivery is receiving increased attention due to the ability of the administration regime to provide a controlled route for the release of an active pharmaceutical ingredient (API) to the systemic circulation. The delivery of drugs, such as testosterone, anastrozole, and HCG, through the skin provides many benefits; primarily, such a means of delivery is a comfortable, convenient and noninvasive way of administering drugs. The variable rates of absorption and metabolism encountered in oral treatments are avoided, and other inherent inconveniences such as gastrointestinal irritation, may be eliminated as well.

Numerous chemical agents have been studied for increasing the rate at which a drug penetrates through the skin. Chemical enhancers are compounds that are administered along with the drug (or in some cases the skin may be pretreated with a chemical enhancer) in order to increase the permeability of the stratum corneum, and therefore provide for enhanced penetration of the drug through the skin. Ideally, such chemical penetration enhancers or “permeation enhancers,” are compounds that are usually innocuous and serve merely to facilitate diffusion of the drug through the stratum corneum. The permeability of many APIs with diverse physicochemical characteristics may be improved using chemical enhancement means. However, there are skin irritation and sensitization problems associated with high levels of certain enhancers.

For the aforementioned reasons, there is a need for a noninvasive way to administer testosterone boosters, such as employing a transdermal composition that includes testosterone, anastrozole, and HCG, synergistically combined; and which may additionally include permeation enhancers, in order to provide consistent delivery of testosterone, anastrozole, and HCG with reduced side effects.

### SUMMARY

A noninvasive way to administer testosterone booster compositions is disclosed. Compositions and methods for testosterone booster transdermal compositions (TBTC) that includes testosterone in synergistic combination with anastrozole, Human Chorionic Gonadotropin (HCG), and permeation enhancers are disclosed. The disclosed TBTC may allow the delivery of APIs and may reduce the risk of undesirable side effects in a patient. TBTC may include permeation enhancer compositions which may enhance the absorption of testosterone, anastrozole, and HCG. Methods for preparing TBTC are also described.

TBTC may be applied on body surface in a daily dose that results in a pharmacologically effective blood concentration of testosterone over a suitable period of time. The dosages may be of from about 25 mg/day to about 500 mg/day of testosterone; from about 0.1 mg/day to about 1 mg/day of anastrozole; and from about 125 U/day to about 500 U/day of HCG. Most suitable dosages may be of from about 50 mg/day to about 120 mg/day of testosterone; from about 0.1 mg/day to about 1 mg/day of anastrozole; and from about 125 U/day to about 500 U/day of HCG. In one embodiment, TBTC may be applied daily for an undetermined extended period of time. In other embodiments, transdermal anastrozole may be applied as prescribed by a doctor, according to the patient's need.

In one embodiment, TBTC may include testosterone, anastrozole, and HCG as APIs and permeation enhancer compositions. Permeation enhancer compositions may include oils native to the Amazon Rainforest such as Pracaxi oil, Plukenetia volubilis seed oil, Inaja oil, and Patauá oil, among others. Amazon Rainforest oils may have essential fatty acids such as behenic acid, and oleic acid which may provide penetration power. Additionally, permeation enhancer compositions may include water, one or more skin lipids, one or more butters having linoleic acid and linolenic acid, and one or more phospholipids, among other components. Physiological lipids, essential fatty acids, and phospholipids may provide penetration power with restorative benefits to the skin. In one aspect of the disclosure liposomes may be produced and may be present in the final permeation enhancer composition within TBTC. Suitable additives, known to those skilled in the art, may be included in TBTC.

In other embodiments, transdermal anastrozole compositions may include organic solvents as transdermal penetration enhancers, such as: caprylic/capric triglycerides (medium chain triglycerides), ethyl alcohol, ethoxy diglycol, dimethyl sulfoxide (DMSO), glycerin, ispropryl myristate, isoproply palmitate, and propylene glycol, among others.

According to one embodiment, amount of testosterone, or a pharmaceutically acceptable salt thereof, in TBTC may be of about 2% by weight to about 20% by weight, most suitable amount may be of about 5% by weight to about 10% by weight; amount of anastrozole included in TBTC may range from about 0.01% by weight to about 0.1% by weight, most suitable amount may be of about 0.01% by weight to about 0.1% by weight; amount of HCG included in TBTC may range from about 125 U/g to about 500 U/g, most suitable amount may be of about 125 U/g to about 500 U/g; and amount of permeation enhancer compositions included in TBTC may range from about 5% by weight to about 50% by weight, most suitable amount may be of about 10% by weight to about 20% by weight.

According to other embodiments, amount of testosterone, or a pharmaceutically acceptable salt thereof, in disclosed TBTC may be of about 2% weight by volume to about 20% weight by volume, most suitable amount may be of about 5% weight by volume to about 10% weight by volume; amount of anastrozole included in TBTC may range from about 0.01% weight by volume to about 0.1% weight by volume, most suitable amount may be of about 0.01% weight by volume to about 0.1% weight by volume; amount of HCG included in TBTC may range from about 125 U/ml to about 500 U/ml, most suitable amount may be of about 125 U/ml to about 500 U/ml; and amount of permeation enhancer compositions included in TBTC may range from about 5% weight by volume to about 50% weight by volume, most suitable amount may be of about 10% weight by volume to about 20% weight by volume.

TBTC may be applied on body surface in a daily dose that results in a pharmacologically effective blood concentration of testosterone over a suitable period of time. The dosages may be of from about 25 mg/day to about 500 mg/day of testosterone; from about 0.1 mg/day to about 1 mg/day of anastrozole; and from about 125 U/day to about 500 U/day of HCG. Most suitable dosages may be of from about 50 mg/day to about 120 mg/day of testosterone; from about 0.1 mg/day to about 1 mg/day of anastrozole; and from about 125 U/day to about 500 U/day of HCG. In one embodiment, TBTC may be applied daily for an undetermined extended period of time. In other embodiments, transdermal anastrozole may be applied as prescribed by a doctor, according to the patient's need.

In one embodiment, producing TBTC may be achieved by mixing ingredients of TBTC in a homogenizer.

Disclosed TBTC may be applied on body surface as ointments, creams, gels, lotions, solutions, and pastes, among other suitable pharmaceutical preparations.

TBTC may be used for reducing symptoms of testosterone deficiency.

Numerous other aspects, features of the present disclosure may be made apparent from the following detailed description.

### DETAILED DESCRIPTION

The present disclosure is here described in detail. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Active Pharmaceutical Ingredient” or “API” refers to a chemical compound that induces a desired effect, and includes agents that are therapeutically effective, prophylactically effective, or cosmeceutically effective.

“Therapeutically effective amount” refers to a nontoxic but sufficient amount of an active pharmaceutical ingredient to provide the desired therapeutic effect.

“Transdermal drug delivery” refers to administration of a drug to the skin surface of an individual so that the drug passes through skin tissue and into the individual's blood stream, therefore providing a systemic effect.

“Body surface” refers to skin.

“Predetermined area of skin” refers to an area of skin through which a drug is delivered. It is intended for application on a defined area of intact unbroken living skin.

“Permeation enhancement” refers to an increase in the permeability of skin to the selected active pharmaceutical ingredient.

“Effective amount of a permeation enhancer” refers to a nontoxic, non-damaging but sufficient amount of the enhancer to provide the desired increase in skin permeability and, correspondingly, the desired depth of penetration, rate of administration, and amount of drug delivered.

“Vehicle” refers to a substance of no therapeutic value that is used to convey an active medicine for administration.

“Viscosity modulating agent” refers to a component of the composition which alters the viscosity of the overall resulting composition.

“Phospholipids” refers to fat-like organic compounds that resemble triglycerides, but have a fatty acid with a phosphate polar group.

“Liposomes” refers to artificially prepared vesicles made of lipid bilayer, and have concentric phospholipid bilayers.

“Butter” refers to a moisturizing product obtained of oils extracted from seeds and nuts. Butters are solid at room temperature, but melt on the skin.

“Lotion” refers to mixed phase or suspension of an API.

### Description

Embodiments of the present disclosure may be directed towards transdermal delivery of testosterone booster compositions. Compositions and methods for producing testosterone booster transdermal compositions (TBTC) that may include permeation enhancers are described. The present disclosure combines testosterone with anastrozole and Human Chorionic Gonadotropin (HCG) in order to reduce symptoms of testosterone deficiency.

### Formulation

TBTC may include testosterone, anastrozole and HCG as active pharmaceutical ingredients (API), permeation enhancers, suitable solvents, at least one viscosity modulating agent and suitable additives.

In some embodiments, various additives, known to those skilled in the art, may be included in TBTC to facilitate the preparation of suitable forms for patient's applications. For example additives may include humectants, pH adjusting agents, preservatives, emulsifiers, occlusive agents, opacifiers, antioxidants, fragrance, colorants, gelling agents, thickening agents, stabilizers, and surfactants, among others.

In one embodiment, TBTC may include a viscosity modulating agent, such as a thickening agent or gelling agent. Concentrations of viscosity modulating agent may be determined by one skilled in the art, depending on the viscosity desired in order to obtain TBTC that may be retained in the vicinity of the area of application for a brief period of time and allow increased uptake of APIs at the site.

In one embodiment, disclosed TBTC may be a true solution. In other embodiments, viscosity of TBTC may be increased in order to obtain a lotion of TBTC. Disclosed TBTC may be applied on body surface as ointments, creams, gels, lotions, solutions, and pastes, among other suitable pharmaceutical preparations.

### APIs

### Testosterone (TRT)

Testosterone is the primary male androgen, playing a vital role in overall male health. Testosterone is essential to the development and maintenance of specific reproductive tissues (testes, prostate, epididymis, seminal vesicle, and penis) and male secondary sex characteristics. Testosterone plays a key role in libido and erectile function and is necessary for the initiation and maintenance of spermatogenesis.

Testosterone also has important functions not related to reproductive tissues. For example, testosterone positively affects body composition by increasing nitrogen retention, which supports lean body mass, muscle size and strength. Testosterone may also act on bone to stimulate bone formation.

When used within the context of the present disclosure the term “testosterone” must be taken to encompass any androgenic steroid that is functional in reducing symptoms of testosterone deficiency. In one embodiment, TBTC may include methyltestosterone or testosterone propionate as testosterone API. In other embodiments, TBTC may include members selected from the group consisting of natural testosterone, testosterone esters, methyltestosterone, methyl testosterone esters, androstenedione, andrenosterone, dehydroepiandrosterone, fluoxymesterone, methandrostenolone , 17α-methylnortestosterone, norethandrolone, dehydrotestosterone, oxymetholone, stanozolol, ethylestrenol, oxandrolone, bolasterone and mesterolone are representative of androgenic steroids. Of this group, most suitable may be testosterone, methyltestosterone and esters thereof. Representative esters include the propionate, phenylacetate, enanthate and cypionate esters of testosterone and methyltestosterone.

In one embodiment, amount of testosterone, or a pharmaceutically acceptable salt thereof, in TBTC may be of about 2% by weight to about 20% by weight, most suitable amount may be of about 5% by weight to about 10% by weight.

In other embodiments, amount of testosterone, or a pharmaceutically acceptable salt thereof, in disclosed TBTC may be of about 2% weight by volume to about 20% weight by volume, most suitable amount may be of about 5% weight by volume to about 10% weight by volume.

### Anastrozole

Anastrozole is the first in a newer class of third generation selective oral aromatase inhibitors. Anastrozole acts by blocking the enzyme aromatase, subsequently limiting the amount of male hormones that are changed into estrogen by the aromatase enzyme, a process called aromatization.

Anastrozole may also be a part of treatment for men suffering from prostate cancer.

The suppression of estrogen, specifically the hormone estradiol, is often necessary for men who have hormone disorders. Elevated levels of the female hormone (estradiol) in men can be manifested in gynecomastia or growth of breasts in males and hypogonadism or the reduced function of the testes. Excess of estradiol can also contribute to increased risk of stroke, heart attack, chronic inflammation, prostate enlargement and prostate cancer. Prescribing anastrozole for men in these situations has shown significant decrease of estradiol levels and, therefore, a decrease in symptoms and risks.

Generally speaking, the primary use of anastrozole for men is to suppress the production of estrogen, the main female sex hormones.

Furthermore, anastrozole has the ability to increase testosterone in the body. Some studies have shown that natural testosterone levels have increased as much as 60% after the use of anastrozole for 7 about days.

Additionally, the use of anastrozole may decrease fat mass, which can also be tied to estrogen levels.

In one embodiment, amount of anastrozole included in TBTC may range from about 0.01% by weight to about 0.1% by weight, most suitable amount may be of about 0.01% by weight to about 0.1% by weight.

In other embodiments, amount of anastrozole included in TBTC may range from about 0.01% weight by volume to about 0.1% weight by volume, most suitable amount may be of about 0.01% weight by volume to about 0.1% weight by volume.

Human Chorionic Gonadotropin (HCG)

Physicians may provide Human Chorionic Gonadotropin (HCG) hormone therapy to increase male fertility (sperm count) when the patient has sufficient naturally produced Follicle-Stimulating Hormone (FSH) to enable the administration of HCG to potentially increase spermatogenesis in hypogonadotropic men deficient in LH and sperm count.

In males, FSH enhances the production of androgen-binding protein by the Sertoli cells of the testes, and is critical for spermatogenesis. FSH regulates the reproductive processes of the human body. Both LH or HCG as a medication substitute for naturally produced LH) and FSH must be present for spermatogenesis.

The purpose of HCG treatment with regard to male infertility is to increase spermatogenesis in hypogonadotropic men deficient in LH who have sufficient FSH. If the patient has insufficient FSH, then they should consult with a reproductive endocrinologist who may administer both FSH and HCG. FSH is available mixed with LH in the form of Pergonal or Menopur, and other more purified forms of urinary gonadotropins, as well as in a pure forms as recombinant FSH (Gonal F, Follistim). Bruce will confirm if we include these 3 paragraphs or change it.

Hypogonadism is a medical term for a defect of the reproductive system that results in lack of function of the gonads (ovaries or testes). The gonads have two functions: to produce hormones (testosterone, estradiol, antimullerian hormone, progesterone, inhibin B), activin and to produce gametes (eggs or sperm). Deficiency of sex hormones can result in defective primary or secondary sexual development, or withdrawal effects (e.g., premature menopause) in adults. Defective egg or sperm development results in infertility.

Spermatogenesis is the process by which male spermatogonia develop into mature spermatozoa. Spermatozoa are the mature male gametes in many sexually reproducing organisms.

Spermatogenesis produces mature male gametes, commonly called sperm but specifically known as spermatozoa, which are able to fertilize the counterpart female gamete, the oocyte, during conception to produce a single-celled individual known as a zygote. This is the cornerstone of sexual reproduction and involves the two gametes both contributing half the normal set of chromosomes (haploid) to result in a chromosomally normal (diploid) zygote.

Spermatogenesis takes place within several structures of the male reproductive system. The initial stages occur within the testes and progress to the epididymis where the developing gametes mature and are stored until ejaculation. The seminiferous tubules of the testes are the starting point for the process, where stem cells adjacent to the inner tubule wall divide in a centripetal direction beginning at the walls and proceeding into the innermost part, or lumen to produce immature sperm. Maturation occurs in the epididymis and involves the acquisition of a tail and hence motility.

Hormonal control of spermatogenesis varies among species. In humans the mechanism are not completely understood, however it is known that initiation of spermatogenesis occurs at puberty due to the interaction of the hypothalamus, pituitary gland and Leydig cells. If the pituitary gland is removed, spermatogenesis can still be initiated by follicle stimulating hormone and testosterone.

Follicle stimulating hormone stimulates both the production of androgen binding protein by Sertoli cells, and the formation of the blood-testis barrier. Androgen binding protein is essential to concentrating testosterone in levels high enough to initiate and maintain spermatogenesis, which can be 20-50 times higher than the concentration found in blood. Follicle stimulating hormone may initiate the sequestering of testosterone in the testes, but once developed only testosterone is required to maintain spermatogenesis. However, increasing the levels of follicle stimulating hormone will increase the production of spermatozoa by preventing the apoptosis of type A spermatogonia. The hormone inhibin acts to decrease the levels of follicle stimulating hormone.

The Sertoli cells themselves mediate parts of spermatogenesis through hormone production. The Leydig cells are also capable of producing estradiol in addition to their main product testosterone.

HCG is a hormone prescribed for men by some physicians to treat male infertility (low sperm count) by functioning as a substitute for the deficient secretion of LH by the pituitary gland. Similar to naturally produced LH, the HCG administered in this treatment stimulates the testes to increase testosterone production and spermatogenesis or the process of creating sperm. This treatment requires that a patient present with a sufficient level of naturally produced FSH. The gonadotropin FSH in combination with HCG induces spermatogenesis in hypogonadotropic men. This treatment does not include administering both recombinant FSH and LH to induce spermatogenesis.

HCG is approved for use in cases of hypogonadotropic hypogonadism (hypogonadism secondary to a pituitary deficiency). It is used to stimulate the testes of men who are hypogonadal or lack sufficient testosterone production.

HCG has been used for temporary improvement or as a “boost” for certain men with borderline or slightly low testosterone levels in order to improve potency and libido.

HCG may also be used in young boys when their testicles have not dropped down into the scrotum normally. Additionally, HCG may be used to increase testicular size after long-term testosterone or anabolic steroid use.

In one embodiment, amount of HCG included in TBTC may range from about 125 U/g to about 500

U/g, most suitable amount may be of about 125 U/g to about 500 U/g.

In other embodiments, amount of HCG included in TBTC may range from about 125 U/ml to about 500 U/ml, most suitable amount may be of about 125 UNI to about 500 U/ml.

Disclosed TBTC may include permeation enhancers which may improve the penetration of testosterone, anastrozole, and HCG in skin, allowing a better absorption of testosterone, anastrozole, and HCG.

### Permeation Enhancers

Permeation enhancer compositions may be added to TBTC at a given percentage to give permeation power to TBTC.

Permeation enhancer composition included in TBTC may be a liquid or semi-liquid that includes phospholipids. Permeation enhancer compositions may include one or more naturally occurring substances, including one or more phospholipids, one or more oils rich in essential fatty acids (behenic acid, and oleic acid), one or more skin lipids, and one or more butters rich in linoleic acid and linolenic acid. The ingredients within permeation enhancer composition may act synergistically to increase the skin permeation to water and oil soluble products. When the permeation enhancer composition is prepared, liposomes may be formed from the fatty acids, including behenic acid and oleic acid that may be present in the one or more oils, and are stabilized by the phospholipids in permeation enhancer composition. More specifically, when permeation enhancer composition is added to water or a water-incorporating composition, liposomes may be formed.

Liposomes may be composed of naturally-derived phospholipids with mixed lipid chains or other surfactants. In some embodiments, the liposomes that are formed may be used to deliver APIs, transdermally to the skin's surface. Liposomes that are formed using embodiments of the present disclosure may be stabilized by the phospholipids.

Many types of phospholipids may be used in embodiments of the present disclosure. In one embodiment, the phospholipids used in permeation enhancer composition may include one or more of phosphatidylcholine, lysophosphotidylcholine, hydrogenated phospholipids, and unsaturated phospholipids. The polar end of the phospholipid molecule is hydrophilic, or soluble in water, and the other end or the fatty-acid end is hydrophobic, or soluble in fats. Phospholipids are ideal compounds for forming the biological membrane. There are two recognized classes of phospholipids, including phosphoglycerids, or those that have a glycerol backbone, and those phospholipids that include sphingosine. Examples of phosphoglycerids may include phosphatidic acid (phosphatidate) (PA), phosphatidylethanolamine (cephalin) (PE), phosphatidylcholine (lecithin) (PC), phosphatidylserine (PS), and phosphoinositides, which further include phosphatidylinositol (PI), phosphatidylinositol phosphate (PIP), phosphatidylinositol bisphosphate (PIP2), and phosphatidylinositol triphosphate (PIP3). Phospholipids that include sphingosine, also termed phosphosphingolipids, may include ceramide phosphorylcholine (sphingomyelin) (SPH), ceramide phosphorylethanolamine (sphingomyelin) (Cer-PE), and ceramide phosphorylglycereol. The most abundant types of phosphoglycerids are phosphatidylcholine (lecithin), phosphatidylethanolamine, phosphatidylserine, phosphatidylinositol, phosphatidylglycerol, and cardiolipin. Lysophospholipids (LPL) have a free alcohol in either the sn-1 or sn-2 positions. The prefix ‘lyso-’ comes from the fact that lysophospholipids were originally found to be hemolytic, but is now used to refer generally to phospholipids missing an acyl chain. LPLs may be the result of phospholipase A-type enzymatic activity on regular phospholipids, such as phosphatidylcholine or phosphatidic acid, although they can also be formed by the acylation of glycerophospholipids or the phosphorylation of monoacylglycerols.

Lysophosphatidylcholine (LPC) has been found to penetrate into the dermis faster than phosphatidylcholine, such that a small amount of LPC may penetrate the skin without damaging skin structure, and may be enzymatically degraded into several lipids.

Other components that may be included in permeation enhancer composition may be oils that are rich sources of essential fatty acids, behenic acid, and oleic acid. The supply of essential fatty acids and antioxidant molecules may restore the cutaneous permeability and the function of the skin barrier. The supply of essential fatty acids and antioxidant molecules may also contribute to the control of the imperceptible water loss and maintain moisture of the skin.

Behenic acid and oleic acid, when used by themselves, may be irritating when applied to the skin, which makes behenic acid and oleic acid difficult to use as permeation enhancers. While having an irritating effect on the skin, behenic acid and oleic acid may also be effective vehicles at delivering APIs through the skin. In one embodiment, oil from a tree in Brazil has the highest natural sources of behenic acid and oleic acid. The tree is called Pentaclethara macroloba, or more commonly termed the Pracaxi tree. Pentaclethra macroloba seed oil, also called Pracaxi oil, is extracted from the tree, which includes high concentrations of behenic acid and oleic acid. Generally, Pracaxi oil may include about 20% of behenic acid and about 35% of oleic acid. In some cases, Pracaxi oil may include more than these percentages. As behenic acid and oleic acid are present in an oil, the effects of the acids may be less irritating on the skin, and as such makes Pracaxi oil a good choice for one of the ingredients of permeation enhancers.

In some embodiments, another oil that may be used, within permeation enhancer compositions, in combination with Pracaxi oil is Plukenetia volubilis seed oil, also known as Inca Inchi. Plukenetia volubilis seed oil is native to the Amazon Rainforest. Plukenetia volubilis seed oil extracted from the Plukenetia volubilis plant is one of the largest plant sources of the Omega family of fatty acids, including a high concentration of protein. Plukenetia volubilis seed oil is also rich in iodine and vitamin A and vitamin E. Plukenetia volubilis seed oil is a natural oil with an exceptional content in polyunsaturated fatty acids (greater than 90%) and tocopherols (1.5 to 2 g/kg). Plukenetia volubilis seed oil is a vegetable oil having both essential fatty acids in such a high amount, including 49% of alphalinolenic acid (omega-3) and 34% of linoleic acid (omega-6). While Plukenetia volubilis seed oil has a very high amount of fatty acids, Plukenetia volubilis seed oil also has high amounts of behenic acid (10-30%) and oleic acid (35-80%). When an oil such as Plukenetia volubilis seed oil and/or Pracaxi oil are used, behenic acid and oleic acid may work to enhance the restoration of cutaneous barrier organization and epidermal elasticity, in addition to contributing to the control of imperceptible water loss, thus, maintaining skin hydration. This is, at least in part, due to the high amounts of essential fatty acids in Plukenetia volubilis seed oil and Pracaxi oil. The link between skin permeation and hydration is clear. Increasing the permeability of the stratum corneum may be achieved by the increase of water content in the stratum corneum. Hydration by occlusion may cause a swelling of the corneocytes and subsequently may increase the skin permeation to APIs.

Still yet another oil that may be included in permeation enhancer compositions may be from a tree called Maximiliana maripa palm, or Inaja. Inaja oil has one of the highest sources of lauric acid (greater than 40%) and oleic acid (greater than 15%). Further, the highest concentration of fatty acids found in the Inaja may be found in kernel oil, as opposed to the pulp oil. Inaja is an indigenous Amazonian palm widespread in the state of Pará, growing around the Amazon River estuary. Oil from Inaja may be extracted from the fruits of the Inaja palm, which may include about 70% short-chain fatty acids, including lauric acid and myristic acid.

In further embodiments other oils such as Buriti, Patauá, Tucuma, Bacuri, Ucuuba, Muru-Muru, Andiroba, and Copaiba, among others, may be included in permeation enhancer composition within disclosed TBTC.

Patauá oil may be extracted from the mesocarp of the patauá palm and generally appears as a greenish-yellow and transparent liquid, with little odor and taste, having the physical appearance and composition of fatty acids that are similar to olive oil (Olea europaea). Patauá oil includes high content of unsaturated fatty acids. Due to high content of oleic acid within patauá oil, patauá oil has moisturizing properties.

Andiroba oil may be extracted from the Carapa guianensis tree. Andiroba oil is rich in omega-3 fatty acid, which may be fast absorbed into the skin. The oil is also a rich source of essential fatty acids, including oleic, palmitic, myristic and linoleic acids, and includes non-fatty components such as triterpenes, tannins, and alkaloids, which may be isolated as Andirobina and Carapina.

Copaiba balsam may be extracted from the bark of the Copaifera officinalis Jacq. tree where copaiba balsam accumulates in cavities within the tree trunk. The chemical composition of the oil-resin of Copaiba may include approximately 72 sesquiterpenes (hydrocarbons) and 28 diterpenes (carboxylic acids), and the oil may include 50% of each of these terpenes.

Ucuúba butter may be obtained from the seeds of the Virola sebifera Aubl tree. Ucuúba butter includes high concentrations of lauric, myrist and palmitic acid, as well as vitamin C and A.

Bacuri (Platonia insignis) is an ornamental tree that may be found in the Amazonian forest, the seeds of which include high oil levels and high percentages of palmitic and oleic fatty acids.

Another component of permeation enhancer composition may be skin lipids. Examples of skin lipids that may be used in permeation enhancer composition may include ceramides and/or squalene. Ceramides are the major lipid constituent of lamellar sheets. Ceramides are a structurally heterogeneous and complex group of sphingolipids including derivatives of sphingosine bases in amide linkage with a variety of fatty acids. Differences in chain length, type, and extent of hydroxylation and saturation are responsible for the heterogeneity of the epidermal sphingolipids. Ceramides may play an important role in structuring and maintaining the water permeability barrier function of the skin. In conjunction with the other stratum corneum lipids, they form ordered structures. A structured semi-occlusive barrier that increases skin hydration may be a positive influence on the penetration of APIs.

Another skin lipid that may be used is squalene, which is a lipid fat in the skin. When used together with a ceramide and a phospholipid, such as phosphatidylcholine, the permeation enhancer composition may be mild such that permeation enhancer composition may be used on even sensitive skin. Squalene may also help to decrease water evaporation, thus speeding up skin permeation to APIs and decreasing irritation made by surfactants found in emulsions.

Yet another component of permeation enhancer composition may be butters rich in linoleic acid and linolenic acid. One example of linoleic acid and linolenic acid rich butters may be butyrospermum parkii butter, also known as shea butter. Other exemplary butters that may be used within permeation enhancer compositions may include cupuacu butter, buriti butter, passionfruit butter, mango butter, tucuma butter, palm butter, murumu butter, chamomile butter, cocoa butter, orange butter, lemon grass butter, avocado butter, tamanu butter, aloe butter, shea butter, monoi butter, pomegranate butter, almond butter, jojoba butter, red palm butter, acai butter, olive butter, matcha green tea butter, brazil nut butter, macadamia butter, kokum butter, mafura butter, coffee butter, tucuma butter, ucuuba butter, bacuri butter, and chamomile butter, among others.

In one embodiment, amount of permeation enhancer compositions included in TBTC may range from about 5% by weight to about 50% by weight, most suitable amount may be of about 10% by weight to about 20% by weight.

In other embodiments, amount of permeation enhancer compositions included in TBTC may range from about 5% weight by volume to about 50% weight by volume, most suitable amount may be of about 10% weight by volume to about 20% weight by volume.

In other embodiments, transdermal anastrozole compositions may include organic solvents as transdermal penetration enhancers, such as caprylic/capric triglycerides (medium chain triglycerides), ethyl alcohol, ethoxy diglycol, dimethyl sulfoxide (DMSO), glycerin, ispropryl myristate, isoproply palmitate, and propylene glycol, among others.

Methods of Elaboration

Various methods may be used to produce disclosed TBTC. In one embodiment, in order to produce TBTC, APIs with permeation enhancers may be mixed in a first vessel. The mixture within first vessel may be at room temperature. Then, mixture within first vessel may be mixed with a suitable base, such as PCCA Lipoderm®, among others. The mixing may then be stopped such that TBTC may be packaged in suitable containers.

Application

Disclosed TBTC, when applied on a body surface, may deliver a therapeutically effective amount of testosterone, anastrozole, and HCG to the systemic circulation of the patient. In particular, TBTC may be used to deliver a predetermined amount of testosterone, anastrozole, and HCG to achieve a predetermined bloodstream level of testosterone, anastrozole, and HCG.

The synergistic effect between testosterone, anastrozole, and HCG may lead to increased levels of testosterone in the patient; therefore, TBTC may be used in treating a wide variety of conditions responsive to testosterone deficiency.

In one embodiment, TBTC may be applied on body surface to restore testosterone and maintain testicular function.

Testosterone deficiency can result from underlying disease or genetic disorders and is also frequently a complication of aging. For example, primary hypogonadism results from primary testicular failure. In this situation, testosterone levels are low and levels of pituitary gonadotropins (LH and FSH) are elevated. Secondary hypogonadism is due to inadequate secretion of the pituitary gonadotropins. In addition to a low testosterone level, LH and FSH levels are low or low-normal. Some of the sequelae of adult testosterone deficiency include a wide variety of symptoms, such as: loss of libido, erectile dysfunction, oligospermia or azoospermia, absence or regression of secondary sexual characteristics, progressive decrease in muscle mass, fatigue, depressed mood and increased risk of osteoporosis. Many of these disorders are generically referred to as male menopause.

In other embodiments, TBTC may be applied on body surface of a patient that is in need of increased muscle mass. TBTC may also be administered to a patient that suffers from lipodystrophy and to a patient that is in need of increased lymphocyte levels. TBTC may also be administered to a patient in need of reduced triglyceride level or to a patient that suffers from benign prostate hypertrophy. Furthermore, TBTC may be administered to a patient that suffers from prostate cancer or to a patient that suffers from a disorder related to male hypogonadism.

The specific type and amount of testosterone may vary depending on the particular disease or condition being treated, and may be added in any concentration required to achieve a particular result. Different formulations may be designed to provide higher or lower testosterone doses.

TBTC may be applied on body surface in a daily dose that results in a pharmacologically effective blood concentration of testosterone over a suitable period of time. The dosages may be of from about 25 mg/day to about 500 mg/day of testosterone; from about 0.1 mg/day to about 1 mg/day of anastrozole; and from about 125 U/day to about 500 U/day of HCG. Most suitable dosages may be of from about 50 mg/day to about 120 mg/day of testosterone; from about 0.1 mg/day to about 1 mg/day of anastrozole; and from about 125 U/day to about 500 U/day of HCG. In one embodiment, TBTC may be applied daily for an undetermined extended period of time. In other embodiments, transdermal anastrozole may be applied as prescribed by a doctor, according to the patient's need.

TBTC may be applied on any suitable area of skin. Most suitable sites to apply TBTC may be ventral forearm, upper arm, and chest. Disclosed TBTC may be applied to those areas of skin which provide maximal systemic absorption due to increased cutaneous blood flow and heat.

In some embodiments, the amount of TBTC administered is a defined, finite amount that provides a therapeutically effective amount (such as a daily dose) of testosterone, anastrozole, and HCG.

The use of disclosed TBTC may lead to increased flux of testosterone as well as anastrozole and HCG, therefore enabling a greater proportion of testosterone, anastrozole, and HCG in a dose to be delivered to the patient and using a smaller area of skin.

In some embodiments, disclosed TBTC may be applied manually with or without an applicator such as a dropper or pipette, an applicator such as a swab, brush, cloth, pad, sponge, or with any other applicator, such as a solid support including paper, cardboard or a laminate material, including material with flocked, glued or otherwise fixed fibers. Alternatively, TBTC may be applied as an aerosol or non-aerosol spray, from a pressurized or non- pressurized container. In further embodiments, TBTC may be administered in metered doses, such as from a metered dose applicator or from an applicator including a daily dose of TBTC.

TBTC may become touch dry within about one to three minutes of application to body surface.

In another aspect of the present disclosure, pharmaceutical compositions including synergistic combination of testosterone with anastrozole and HCG may include other dosage forms in addition to transdermal; such as oral capsules, sublingual drops, tablet triturates or troches, and injection solutions, among others, employing suitable vehicles.

### EXAMPLES

Example #1 is an embodiment for formulation of TBTC which includes the ingredients described in table 1:

TABLE 1
illustrates example#1 TBTC.

| INGREDIENTS | AMOUNT |
|---|---|
| Anastrozole 2%/Microcrystalline Cellulose | 0.5-5% |
| Trituration | |
| Chorionic Gonadotropin 10,000 U/0.1 Gm/ | 0.125-0.5% |
| Mannitol Trituration | |
| Testosterone USP Micronized | 2-20% |
| Transdermal Penetration Enhancers | 5-50% |
| Base | q.s. 100% |

Example #2 is an embodiment for formulation of TBTC which includes PCCA Lipoderm® as base, described in table 2:

TABLE 2
illustrates example #2 TBTC.

| INGREDIENTS | AMOUNT | |
|---|---|---|
| Testosterone USP Micronized | 1.2-12 | g |
| Anastrozole 2%/Microcrystalline Cellulose | 0.3-3 | g |
| Trituration | | |
| Chorionic Gonadotropin 10,000 U/0.1 Gm/ | 0.075-0.3 | g |
| Mannitol Trituration | | |
| Propylene Glycol USP | 3-5 | ml |
| Base, PCCA Lipoderm ® | q.s. 60 | ml |

While various aspects and embodiments have been disclosed, other aspects and embodiments are contemplated. The various aspects and embodiments disclosed are for purposes of illustration and are not intended to be limiting, with the true scope and spirit being indicated by the following claims.

1. A method of increasing hormone levels comprising: administering to a patient a pharmaceutical composition comprising testosterone, anastrozole, Human Chorionic Gonadotropin (HCG), and at least one permeation enhancer.

2. The method according to claim 1, wherein the testosterone is applied at about 25 mg/day to about 500 mg/day.

3. The method according to claim 1, wherein the anastrozole is applied at about 0.1 mg/day to about 1 mg/day.

4. The method according to claim 1, wherein the HCG is applied at about 125 U/day to about 500 U/day of HCG.

5. The method according to claim 1, wherein the administration of the pharmaceutical composition is to the skin.

6. The method according to claim 1, wherein the permeation enhancer is selected from the group comprising pracaxi oil, Plukenetia volubilis seed oil, Inaja oil, and Patauá oil, and combinations thereof.

7. The method according to claim 1, wherein the permeation enhancer is selected from the group comprising behenic acid, oleic acid, and combinations thereof.

8. The method according to claim 1, wherein the permeation enhancer further comprises water, one or more skin lipids, one or more butters having linoleic acid or linolenic acid, and one or more phospholipids, and combinations thereof.

9. The method according to claim 1, wherein the permeation enhancer comprises at least one medium chain triglyceride, ethyl alcohol, ethoxy diglycol, dimethyl sulfoxide, glycerin, ispropryl myristate, isoproply palmitate, propylene glycol, and combinations thereof.

10. The method according to claim 9, wherein the at least one medium chain triglyceride comprises at least one from the group consisting of caprylic triglyceride, capric triglyceride, and combinations thereof.

11. The method according to claim 1, wherein the testosterone comprises about 2% to about 20% by weight of the pharmaceutical composition.

12. The method according to claim 1, wherein the testosterone comprises about 5% to about 10% by weight of the pharmaceutical composition.

13. The method according to claim 1, wherein the anastrozole comprises about 0.01% to about 0.1% by weight of the pharmaceutical composition.

14. The method according to claim 1, wherein the pharmaceutical composition comprises about 125 U/g to about 500 U/g of HCG.

15. The method according to claim 1, wherein the permeation enhancer comprises about 5% to about 50% by weight of the pharmaceutical composition.

16. The method according to claim 1, wherein the permeation enhancer comprises about 10% to about 20% by weight of the pharmaceutical composition.

17. The method according to claim 1, wherein the pharmaceutical composition is formulated as an ointment, cream, gel, lotion, solution, or paste.

18. A pharmaceutical composition, comprising: testosterone, anastrozole, Human Chorionic Gonadotropin (HCG), and at least one permeation enhancer.

19. The pharmaceutical composition of claim 18, wherein the testosterone comprises about 2% to about 20% by weight of the pharmaceutical composition.

20. The pharmaceutical composition of claim 18, wherein the testosterone comprises about 5% to about 10% by weight of the pharmaceutical composition.

21. The pharmaceutical composition of claim 18, wherein the anastrozole comprises about 0.01% to about 0.1% by weight of the pharmaceutical composition.

22. The pharmaceutical composition of claim 18, wherein the pharmaceutical composition comprises about 125 U/g to about 500 U/g of HGC.

23. The pharmaceutical composition of claim 18, wherein the permeation enhancer comprises about 5% to about 50% by weight of the pharmaceutical composition.

24. The pharmaceutical composition of claim 18, wherein the permeation enhancer comprises about 10% to about 20% by weight of the pharmaceutical composition.

25. The pharmaceutical composition of claim 18, wherein the pharmaceutical composition is formulated as an ointment, cream, gel, lotion, solution, or paste.

26. The pharmaceutical composition of claim 18, wherein the permeation enhancer is selected from the group comprising pracaxi oil, Plukenetia volubilis seed oil, Inaja oil, and Patauá oil, and combinations thereof.
